(19) United States
(12) Patent Application Publication
(10) Pub. No.: US 2012/0094910 A1
(43) Pub. Date: Apr. 19, 2012

(54) IMPROVED PROCESS FOR THE PREPARATION OF DESMOPRESSIN OR ITS PHARMACEUTICALLY ACCEPTABLE SALTS

(76) Inventors: Ananda Kuppanna, Hyderabad (IN); Mallikarjuna Sarma Dokka, Hyderabad (IN); Bulliraju Kamana, Hyderabad (IN); Sreelatha Vanjivaka, Hyderabad (IN); Debashish Datta, Hyderabad (IN)

(21) Appl. No.: 13/263,123

(22) PCT Filed: Apr. 5, 2010
(86) PCT No.: PCT/IN10/00222

§ 371 Date: Oct. 6, 2011

(30) Foreign Application Priority Data
Apr. 6, 2009 (IN) 794/CHE/2009

(51) Int. Cl. A61K 38/11; C07K 7/16

(52) U.S. Cl. 514/10.9; 530/315

(57) ABSTRACT

The present invention relates to a novel and improved process for the preparation of 1-deamino-8-D-arginine vasopressin (Desmopressin) or its pharmaceutically acceptable salts thereof and also relates to an improved process for the purification of Desmopressin or its pharmaceutically acceptable salts. Further, the present invention also relates to pharmaceutical composition of Desmopressin or its pharmaceutically acceptable salts thereof.

This application claims priority to Indian patent application No. 794/CHE/2009 filed on Apr. 6, 2009, the contents of which are incorporated by reference in their entirety

### FIELD OF THE INVENTION

The present invention relates to a novel and improved process for the preparation of 1-deamino-8-D-arginine vasopressin (Desmopressin) or its pharmaceutically acceptable salts and further relates to a pharmaceutical composition comprising the same.

### BACKGROUND OF THE INVENTION

Arginine Vassopressin (AVP) which modulates antidiuretic activity and specificity led to the synthesis of desmopressin, a peptide possessing high antidiuretic activity, specificity and increased duration of action. Structurally, it is an analog of naturally occurring arginine vasopressin, in which the terminal amino group is removed and the amino acid residue Arg8 is replaced by DArg.

Desmopressin is a predominant harmone analog of Vasopressin and is shown to have antidiuretic effect that decreases urinary volume and increases urine osmolality. Desmopressin is a therapeutic peptide widely used for treating diabetes insipidus, primary nocturnal enuresis, hemophilia and type 1 Von Willebrand's disease. It also has the ability to improve human memory functions. Desmopressin is marketed in United States as DDAVP, MINIRIN, and STIMATE. Desmopressin is a cyclic nona-peptide with a disulphide bridge and is chemically known as 1-deamino-8-D-arginine vasopressin having the following structure.

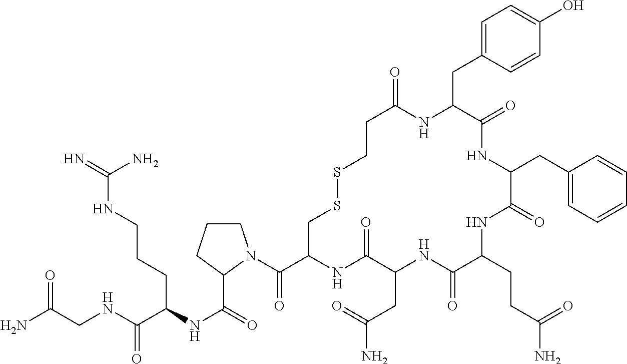

Desmopressin can be represented in terms of chemical formula as follows

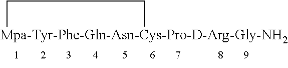

The synthesis of peptides is generally carried out through condensation of the carboxylic group of one amino acid and amino group of another amino acid to form a peptide bond. Peptide sequence can be constructed by repeating the condensation of the individual amino acids in stepwise elongation, or by condensation between two or more pre-formed peptide fragments. In both types of condensation, the amino and carboxyl groups that are not desired to participate in the reaction must be blocked/protected with protecting groups. In addition, reactive side chain functionalities of the amino acids also need to be protected.

The synthesis of peptides has been described by two general methods in the literature. The first method is a solution phase procedure, based on fragment condensation. The process comprises removing a protecting group and coupling with another amino acid using fragment strategy. The process involves a time consuming, multi step synthesis, and presents additional problems during the separation.

According to the process known in the art, isolation of each step in building up the amino acid chain results in decrease of yield and also more workups are needed. In homogenous phase synthesis of peptide, repeated purification between individual steps is required, which may result in pure product but with low yield. The disulfide bridge formation is occurred on oxidation with potassium ferricyanide resulting in the formation of impurities and requires repeated purifications to get pure product thereby resulting in low yield.

The second method for the synthesis of peptides utilizes the entire peptide chain using solid phase peptide synthesis.

In conventional solid phase peptide synthesis, the peptide-resin linkage is critical to the synthetic procedure. The linkage must be appropriately stable during the deprotection of the amino blocking/protecting groups. If the linkage is not stable during the deprotection conditions, the peptide will be cleaved prematurely from the resin. Further, the linkage must be cleaved readily upon completion of the synthesis of the peptide, preferably under conditions that will not damage the peptide being recovered. Hence, a balance between the resin peptide linkage retention during deprotection of amino group and cleavage of completely synthesized peptide poses an opportunity of appropriate selection of resin, deprotecting agent, cocktail composition for cleavage of the resin from peptide and global deprotection of linked amino acids in order to arrive at an improved process.

Initial methods however, the benzyl group has been utilized for the protection of the sulfhydryl group of Cys followed by treatment of fully protected peptide with a sodium liquid ammonia and subsequent oxidation to form the disulphide bridge. The main disadvantage of such an approach has resulted in low yield of pure material obtained in the final cyclization step.

Desmopressin is first disclosed in U.S. Pat. No. 3,497,491. This patent discloses a homogenous method (Solution phase). Desmopressin is prepared by building the amino acid chain by isolation of each step and deprotecting the protecting groups using alkali metal in liquid ammonia. Disulfide bridge is formed by using oxidizing agent such as potassium ferricyanide in an aqueous solution at pH at 6.5-7 to form Desmopressin.

U.S. Pat. No. 5,200,507 discloses a process for the preparation of Desmopressin using solid phase peptide synthesis (MBHA Resin). U.S. Pat. No. '507 discloses method of separating a peptide from a resin to which the peptide is bound comprising treating the resin bound peptide with hydrogen fluoride so as to cleave the peptide. According to this process, the preparation of Desmopressin involves use of strong acid such as HF at −5 to 0° C. The cleavage of peptide using HF results in formation of impurities. Moreover, use of HF in plant scale is difficult and highly corrosive.

U.S. Pat. No. 5,596,078 discloses a process for the preparation of Desmopressin at relatively high purity using oxidation in presence of iodine dissolved in protic solvent, which in-turn is purified by adding the mixture containing cyclic peptide using cation exchange resin. The present process doesn't seem to be cost effective.

US 20040249121 A1 discloses a process for the preparation of Desmopressin using solid phase peptide synthesis comprising protected linear peptide having at least two protected thiol containing residues of which at least one thiol containing residue is protected with an orthogonal protecting groups, which on reaction with acidic composition gives semi-protected linear peptide with protecting groups on thiol containing residues. The semi protected linear peptide is purified and oxidized to give a cyclic peptide, which on purification gives Desmopressin. This process involves more number of purification steps for obtaining pure Desmopressin which results in decrease of yield and increase in cost of the product.

US 20060148699 A1 discloses a process for the preparation of peptide by solid phase peptide synthesis using rink amide resin loaded with 0.7 mmol/g followed by two step purification for the preparation of pure Desmopressin. The two step purification in the synthesis results in decrease of yield.

There is no sufficient data available in the literature for the preparation of Desmopressin such as particle size of the resin to be used, loading capacity of the resin and physical characterization data.

Therefore, there exists a need to develop an alternate and improved process for the preparation of Desmopressin with improved yield. Further the process involved should be simple, convenient and cost-effective for large scale production.

The present invention overcomes the prior art deficiencies. The advantage of present invention includes using high load resin over low load resin resulting in improved yield, less workups and small size of the vessel to give a peptide in required yield and purity.

### OBJECT OF THE INVENTION

The main object of the invention is to provide an improved process for preparation of Desmopressin.

The main object of the invention is to provide an improved process for preparation of Desmopressin using high load resin.

Another object of the present invention is to provide process for the purification of peptide of formula II in presence of organic solvent or mixture thereof.

Another object of the present invention is to provide process for the purification of peptide of formula II in presence of organic solvent or mixture thereof.

Yet another object of the present invention is to provide an improved process for the purification of Desmopressin.

### SUMMARY OF THE INVENTION

It is a principal aspect of the present invention to provide an improved process for the preparation of Desmopressin or its pharmaceutically acceptable salts which comprises coupling of amino acids optionally having protective groups in peptide sequence to give a compound of formula (III) which is cleaved from the resin with simultaneous deprotection of protective groups to give a crude compound of formula II, which is further purified and oxidized to give a Desmopressin and finally converted to its pharmaceutically acceptable salt.

In another aspect of the present invention, there is provided an improved process for the purification of linear peptide of formula II in presence of an organic solvent or mixture thereof.

In another aspect of the present invention, there is provided improved process for the purification of Desmopressin by loading in to preparative HPLC and eluting with buffer containing acetic acid to give pure Desmopressin acetate.

In yet another aspect, the present invention provides pharmaceutical composition of Desmopressin or its pharmaceutically acceptable salts thereof.

### DETAILED DESCRIPTION OF THE INVENTION

The present invention relates to a novel and improved process for the preparation of 1-deamino-8-D-arginine vasopressin (Desmopressin) or its pharmaceutically acceptable salts and further relates to a pharmaceutical composition comprising the same.

For the purpose of clarity and as an aid in the understanding of the invention, as disclosed and claimed herein, the following terms and abbreviations are defined below
AcOH acetic acid
tBu tert-butyl
DCC N,N′-dicyclohexyl carbodiimide
DCM dichloromethane
DIC N,N′-diisopropylcarbodiimide
DMF N,N′-Dimethylformamide
Fmoc 9-fluorenylmethoxycarbonyl
HOBt N-hydroxybenzotriazole
Mpa mercaptopropionic acid
MTBE Methyl tert-butyl ether
Pbf pentmethyldihydrobenzofuransulfonyl
RT room temperature
SPPS solid phase peptide synthesis
TFA trifluoroacetic acid
TIS triisopropylsilane
Trt trityl

As used herein, the term ‘Capping’ relates to a process of protecting free functional groups on the polymeric resin as acetyl or ester form.

The term “pharmaceutical compositions” as used herein refers to dosage form for oral administration in the form of tablets, capsules, pills, powders, granules, particles, pellets, beads, or mini-tablets. Preferred dosage forms are tablets.

The excipients included in the composition are those which are customary and known to a person skilled in the art. These include without any limitations, diluents, fillers, binders, disintegrants, surfactants, stabilizers, glidants, lubricants etc.

The tablets can be prepared by conventional processes known to a person skilled in the art. Preferably, processes employed for the preparation of the tablets according to the invention include wet granulation, dry granulation and/or direct compression.

In one embodiment, the present invention provides process for the preparation of Desmopressin comprising the steps of:
a) swelling or soaking of the resin in a solvent,
b) deprotecting the Fmoc group of swelled resin,
c) anchoring first protected terminal amino acid to a resin,
d) capping the resin obtained in step c),
e) selective deprotection of the amino group,
f) coupling carboxyl terminus of the next N-protected amino acid to the amine group,
g) repeating steps e) and f) to form a peptide sequence,
h) cleaving the peptide from the resin and optionally deprotecting the functional protecting groups using a cocktail mixture to obtain dithiol derivative,
i) optionally purifying the dithiol derivative obtained in step h),
j) oxidizing the dithiol derivative to form disulphide bridge to obtain crude Desmopressin,
k) passing the crude Desmopressin through ion exchange resin,
l) purifying the crude Desmopressin to isolate pure Desmopressin acetate.

The process for the preparation of Desmopressin is summarized in synthetic scheme-I depicted below:

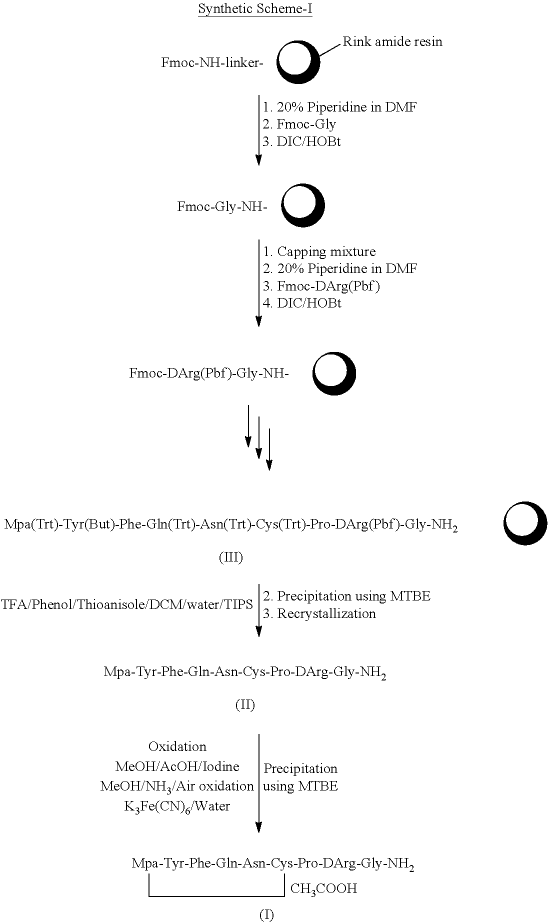

According to the present invention, the different types of resins suitable for the peptide synthesis and their loading capacity, particle size and formation of matrix are listed in the table below. The below listed resins should not be taken as a limitation to the experiment.

| | Particle | | |
|---|---|---|---|
| | size | | |
| Resin | (μm) | Matrix | Loading |
| Tenta gel | 90 | Poly(oxyethylene)-RAM | 0.24 mmol/g |
| SRAM | | Polymer bound | |
| Rink amide | 100-200 | Amino methyl | 1.1 mmol/g |
| resin | | polystyrene crosslinked | |
| | | with 1% DVB | |
| Rink amide | 100-200 | Amino methyl | 0.43 mmol/g |
| resin | | polystyrene crosslinked | |
| | | with 1% DVB | |
| 2-Chlorotrityl | 100-200 | Polystyrene crosslinked | 0.7-0.9 mmol/g |
| chloride | | with 1% DVB | |
| resin (2-CTC) | | | |

The resin used for the preparation of Desmopressin acts as support material and is selected from Tenta gel SRAM, 2-Chlorotrityl chloride resin (2-CTC), Rink amide resin and Rink amide resin (1.1 mmol/g). The selection of polymeric support and attached linker is very critical for overall outcome of the solid phase peptide synthesis. TentaGel is found to be very effective for the preparation of Desmopressin and are comprising of grafted copolymers consisting of a low cross linked polystyrene, but the loading capacity is 0.43 mmol/g.

The Rink amide resin is used in the process of Desmopressin in high load in 1.1 mmol/g with amino methyl polystyrene type linker has additional advantages over the other resins. The advantage of using high load resin is that significantly more peptide for unit measure of beads could be produced with high load resins. This is a consequence of the fact that higher concentrations of reagents and reactants can be achieved with high load resins. Smaller vessel sizes could be employed to generate a given amount of peptide and at least 50% less wash solvents needed while using high loaded resins. For the scale up of the solid phase attractive it is important to reduce the large amounts of reagents typically employed in solid phase peptide synthesis.

The resin used for the preparation of Desmopressin is suspended in an organic solvent to swell. The amount of protected amino acid used in the anchoring step is normally in excess molar quantities and can range from about 2M to 5M with respect to the resin loading capacity, preferably 3 moles of Fmoc-glycine used. The organic solvent utilized for swelling or soaking the resin may selected from methylene chloride, N,N-Dimethylformamide or N-Methylpyrrolidone. The process can optionally be repeated with the solvent system selected. The Fmoc group from the resin was removed by treating with 20% piperidine in DMF.

According to the present invention, the swelled and deprotected resin is then treated with protected first amino acid in presence of organic coupling agent for a desired period of time to affect the coupling. The first amino acid anchored to the resin is typically glycine, wherein the amino terminus of glycine is blocked by a protecting group. Fmoc is used as the protecting group for blocking the amino terminus of glycine.

The coupling reaction may be carried out in a suitable solvent. The solvent that may be used in the coupling step include but are not limited to dichloromethane, dimethylformamide, N-methylpyrrolidone or mixtures thereof. The temperature at which the coupling reaction is carried out may range from about 15° C. to about 40° C.

After completion of the reaction, the resin may be optionally washed with solvents such as dichloromethane, dimethylformamide to remove residual reagents and byproducts formed. The process may be repeated if desired.

Before proceeding for the next steps after anchoring the first protected terminal amino acid, the un-reacted linkers on the resin (polymer) are desired to be appropriately protected in order to avoid the undesired peptide chain formation. Preferably, the free amine functional groups on the polymeric resin may be protected as acetyl. This process is referred to as capping and may be carried out after anchoring the first amino acid to the resin by using acetic anhydride in presence of pyridine and a suitable solvent. The suitable solvent for capping is selected form dichloromethane, chloroform.

According to the present invention, after capping deprotection of the protected amino acid attached to the resin is done selectively in the presence of nucleophilic base such as 20% piperidine in dimethylformamide, methylene chloride or N-methyl pyrrolidine.

The process of selective deprotection further comprises washing the deprotected amino acid with a suitable solvent such as dichloromethane or dimethylformamide or their mixture to remove residual reagents and byproducts formed.

The deprotected amino acid anchored on the resin is coupling with the carboxyl terminus of the next N-protected amino acid to synthesize the desired peptide sequence.

The coupling efficiency after each coupling step may be monitored during synthesis by means of a Kaiser test or any other suitable test. The individual coupling steps, if showing low coupling efficiency may also be repeated prior to proceeding for deprotection and coupling with next amino acid of the sequence.

The amount of protected amino acid used in the coupling step is normally in excess molar quantities and may range from about 1 to about 6 molar equivalents, per molar equivalent of resin with respect to resin loading capacity, preferably, about 1.5 to about 2.0 molar equivalents.

Suitable coupling agents include, but are not limited to BOP, PyBOP, DIC, HBTU or mixture thereof. The amount of coupling agents used may range from about 1 to about 5 molar equivalents, per molar equivalent of resin with respect to resin loading capacity. Preferably, 2 molar equivalents of individual coupling agents per molar equivalent of resin with respect to resin loading capacity may be used.

The steps of the deprotecting the Fmoc group of the amino acid and coupling the next suitably protected amino acid of the sequence may be carried to get the desired desmopressin peptide sequence.

The solvents selected for the coupling reaction is selected from group comprising of dichloromethane, tetrahydrofuran, dimethylformamide, N-methylpyrolidone or mixtures thereof.

The resin after the completion of the reaction is optionally washed with solvents such as DMF and DCM to remove residual reagents and byproducts formed. The process is repeated if desired.

The functional group present on the amino acids used in the process of the present invention may be appropriately protected to avoid any undesired by-products. Suitable protecting groups are described in the literature (See for example, P Wuts, and T. W. Greene, Protective Groups in Organic Synthesis John Wiley & Sons, 4^{th} Edition 2007). The protecting group may vary depending upon the particular amino acid which may include but are not limited to Pbf, tBu or Trt.

According to the present invention, after desired peptide prepared, the cleavage of the peptide from the resin and deprotecting the functional protecting groups on amino acid groups is carried out by using a cocktail mixture. The cleavage of the peptide from the resin not only involves cleavage of peptide but also involves global deprotection (a process for deprotecting the protected amino acid in the peptide, which has additional functional groups). Cleaving the peptide from the resin involves treating the protected peptide anchored to the resin with an acid having at least one scavenger. The acid utilized in the cleavage reagent is TFA. The amount of TFA used for the purpose of cleavage of peptide from the resin and global deprotection in the cocktail mixture may range from 80-90%. The scavengers used are selected from TIS, phenol, thioanisole, water or in any combination thereof.

The two Cocktail mixture for the cleavage of the peptide from resin are disclosed in the prior art and they are.
a) TFA/EDT/Thioanisole/DCM/TIPS in the ratio of about 80%/5%/5%/5%/5% or 80%/5%/3.5%/3.5%/8% respectively or reagent K and
b) TFA/phenol/Thioanisole/DCM/TIPS/H₂O in the preferred volumes of 80%/5%/3.33%/3.33%/5%/3.33% respectively or 80%/5%/3.5%/3.5%/8% respectively.

However, it does not disclose the use of the above conditions for the preparation of Desmopressin.

The Cocktail mixture used for the cleavage of the peptide from resin and as well as global deprotection of the cleaved peptide to get crude Desmopressin thiol is comprising TFA/phenol/Thioanisole/DCM/TIPS/H₂O in a ratio of about 80%/5%/3.33%/3.33%/5%/3.33%.

The solvent used in the cleavage step of the process of the present invention may be selected from dichloromethane, chloroform. The temperature at which the cleavage and global deprotection carried out to isolate the crude Desmopressin thiol is ranging from about 15-40° C., preferably, about 25-30° C.

According to the present invention, after completion of the reaction, the reaction mixture may optionally be filtering and washing with an acid or an organic solvent. The isolation may be carried out by adding an ether solvent to the reaction mass or by adding the reaction mass to the ether solvent selected, preferably, the reaction mass is added to an ether solvent. More preferably, the reaction mass is added to an ether solvent pre-cooled to a temperature of about −5° C. to 5° C. The ether solvent is s selected from group comprising of diethyl ether, diisopropyl ether, t-butyl methyl ether, t-butyl ethyl ether, isopropyl ether or mixture thereof.

The obtained precipitate may be separated by filtration. The obtained crude product may be optionally washed with an organic solvents preferably MTBE and subjected to drying under continuous nitrogen purging.

The isolated Desmopressin thiol (II) is subjected to further purification by using recrystallisation or slurring to get the pure product. The solvent used for purification is selected from ethyl acetate, methanol, ethanol, 1-propanol, 2-propanol or mixtures thereof.

According to present invention, the peptide thiol of formula (II) is subjected to oxidation using methanol/iodine, air oxidation or K₃Fe(CN)₆ to get crude Desmopressin. The peptide formed after oxidation is subjected to decolourization and purification by passing through weak anion exchange resin. The isolation of Desmopressin is carried by evaporating methanol and precipitating with ether solvent to get Desmopressin as a solid. Ether solvents that are used include but are not limited to diethyl ether, t-butylmethyl ether, isopropyl ether or combinations thereof.

According to the present invention, the present invention provides an improved process for the purification of desmopressin which comprises the steps of:
1. Purification by gradient method on preparative HPLC using Novasep column.
2. Isolating pure desmopressin.

The purification process of desmopressin is carried out on preparative HPLC, wherein often a C-18 or C-8 is utilized on reverse phase. The process of present invention utilizes a C-18 column reverse phase column resulted in highly pure desmopressin.

Purification of crude desmopressin is carried out in a gradient method, by elution with a gradient comprising of buffer A: Water/acetic acid and buffer B: methanol/acetic acid.

The desmopressin was eluted at around 30% methanol. During the elution, fractions are collected at regular intervals. The collected fractions are assayed by HPLC to determine the purity and fractions with desired purities may be pooled together.

The purification achieved by this method utilizes the desired salt in a single purification step avoids the additional desalting step.

The purified Desmopressin pooled fractions is then subjected to evaporation to remove methanol solvent. The concentrated pure pooled fraction so obtained may be subjected to Lyophillization under set of parameters of Lyophilization to collect the lyophilized powder which may assayed by purity method of HPLC to ensure that it meets.

Desmopressin obtained by the process of the present invention is analyzed for purity by HPLC. HPLC measurements of Desmopressin samples for chemical purity were performed using Waters system equipped with Purosphere star, 100 RP-C18, 150 mm×4.0 mm, 5 μm using the following mobile phase.
Mobile phase A: Mix 900 Ml of pH 7.0 buffer and 100 mL of acetonitrile.
Mobile phase B: Mix 70 mL of pH 7.0 and 300 ml of acetonitrile.

HPLC chromatogram obtained by the above analytical method of the present invention revealed that Desmopressin contains impurities at relative retention time (RRT) of about 0.93, 0.96 and 1.07 having the content less than 0.5%.

In yet another embodiment, the present invention provides pharmaceutical composition of Desmopressin or its pharmaceutically acceptable salts thereof.

The following examples are provided for illustrative purposes only and are not intended to limit the scope of the invention in any way.

### Example-1

### Preparation of Mpa-Tyr-Phe-Gln-Asn-Cys-Pro-DArg-Gly-NH₂ (Desmopressin Precursor) by SPPS Method

Synthesis of the peptide was carried out by a regular stepwise Fmoc SPPS procedure starting from Rink amide resin (10 g, 10 mmol loading 1.0 mmol/g). The resin was swelled in a dichloromethane (50 mL) for about 2 hours later in DMF (50 mL) for 2 hours. The Fmoc group from the resin was removed by treating with 20% piperidine in DMF. The first amino acid (Fmoc-Gly) was loaded on the resin by a regular coupling procedure. After the coupling of the first amino acid onto the resin, the resin was capped with a capping mixture (acetic anhydride/pyridine/DCM). The Fmoc protecting group was removed with 20% piperidine in DMF. The second amino acid (Fmoc-D-Arg (Pbf)) was introduced to continue amino acid sequence elongation. The Fmoc protected amino acids were activated in situ using HOBt (2.7 g, 20 mmol) and DIC (2.5 g, 20 mmol) in presence of DMF (10 mL). The completion of the coupling was indicated by ninhydrine test. After washing the resin, the Fmoc protecting group on the α-amine was removed with 20% piperidine in DMF. These steps were repeated each time with another amino acid according to peptide sequence. All amino acids used were Fmoc-N^{α} protected except the last building block in the sequence, Trt-Mpa. Trifunctional amino acids were side chain protected as follows: Arg (Pbf), Asn (Trt), Gln (Trt), Cys (Trt) and Tyr (tBu). At the end of the synthesis, the peptide-resin was washed with DCM followed by DMF, methanol and ether respectively and finally vacuum dried to get 30 g of peptide resin.

The cleavage of the peptide from the resin with simultaneous deprotection of the protecting groups was done by treating with TFA/Phenol/Thioanisole/DCM/water/TIPS in the preferred volumes of 80%/5%/3.33%/3.33%/3.33%/5% respectively or 80%/5%/3.5%/3.5%/8% respectively at room temperature for 2 hours. The cleavage mixture was collected by filtration. The resin was washed with trifluoroacetic acid and dichloromethane. The product was precipitated by the addition of 10 volumes of methyl t-butyl ether to the filtrate. The precipitate was filtered and washed several times with methyl t-butyl ether followed by recrystallization from a mixture of ethyl acetate-ethanol (95/5) to obtain 9.0 g of Mpa-Tyr-Phe-Gln-Asn-Cys-Pro-DArg-Gly-NH₂ (II). It was identified by RP-HPLC and LC-MS and purity of the compound was found to be ˜95%.

### Example-2

### Purification of Mpa-Tyr-Phe-Gln-Asn-Cys-Pro-DArg-Gly-NH₂ (II) (Desmopressin Precursor)

Mpa-Tyr-Phe-Gln-Asn-Cys-Pro-D-Arg-Gly-NH₂ (I)

Peptide thiol of formula II (5 g) was slurried in a mixture of ethyl acetate:ethanol (95:5) at 0° C. for 1 hour. The reaction mass was filtered and washed with ethyl acetate to afford 4.5 g of pure peptide thiol of formula II (˜95%).

### Example-3

### Preparation of Desmopressin (Disulphide Bridge Formation)

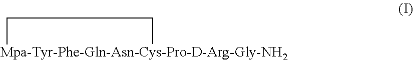

Peptide thiol of formula II (1.0 g) obtained in example-I was dissolved in 1% acetic acid in methanol (400 mL) and then slowly added iodine solution in methanol till the yellow color persists. The reaction mass was stirred for 2 hours at room temperature and the disulphide bridge formation was monitored by using HPLC. The reaction mass was concentrated and the product was precipitated by addition of methyl t-butyl ether, washed several times with methyl t-butyl ether and dried. It was further purified by using anion exchange resin.

### Example-4

### Preparation of Desmopressin (Disulphide Bridge Formation)

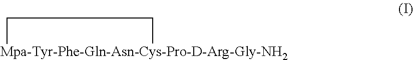

Peptide thiol of formula II (1.0 g) obtained in example-I was dissolved in methanol (400 mL). The pH of the reaction mass was adjusted to 8.0 by using ammonia solution and stirred at 0° C. for 3 hours. The disulphide bridge formation was monitored by using HPLC. Methanol was evaporated from the reaction mass and the product was precipitated by the addition of methyl t-butyl ether, washed several times with methyl t-butyl ether and dried.

### Example-5

### Preparation of Desmopressin (Disulphide Bridge Formation)

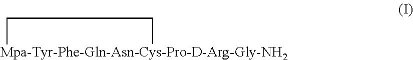

Peptide thiol of formula II (1.0 g) obtained in example-I was dissolved in water (400 mL) and the pH of the reaction mass was adjusted to 8.0 by using ammonia solution. K₃Fe(CN)₆ (0.01 M) was added slowly till the yellow color of the reaction mass persists. The reaction mass was stirred at room temperature for 3 hours. The disulphide bridge formation was monitored using HPLC. The obtained mass was passed through ion exchange resin and lyophilized.

### Example-6

### Purification of Desmopressin

The crude Desmopressin was loaded on to preparative C18 column (50×250 mm, 300 A°). The peptide was purified using aqueous acetic acid (0.05%) and methanol. The pure fractions containing the Desmopressin were pooled. The methanol was evaporated and the aqueous layer was lyophilized to obtain the Desmopressin as white solid. The purified Desmopressin was analyzed by RP-HPLC and mass determined by mass spectrometer.

### Example-7

### Purification of Desmopressin Using Preparative HPLC Column

The crude Desmopressin was loaded on to preparative C-18 column (50×250 mm, 10μ, 300 A°). The peptide was purified by using aqueous phase (Buffer A 0.05% acetic acid/TFA) and methanol/acetonitrile (Buffer B 0.05% acetic acid/TFA) to get pure Desmopressin acetate.

### Example-8

### Preparation of Desmopressin Composition

### Unit Composition:

| | | | |
|---|---|---|---|
| S. | | mg/ | % |
| No. | Ingredients | dosage | w/w |

Intra-granular

| | | | |
|---|---|---|---|
| 1 | Desmopressin Acetate | 0.200 | 0.10 |
| 2 | Mannitol | 113.500 | 56.75 |
| 3 | Copovidone | 6.000 | 3.00 |
| 4 | Starch | 76.400 | 38.20 |
| 5 | Dehydrated Alcohol | q.s. | — |

Extra- granular

| | | | |
|---|---|---|---|
| 6 | Colloidal Silicon Dioxide | 0.400 | 0.20 |
| 7 | Talc | 2.000 | 1.00 |
| 8 | Sodium Stearyl fumarate | 1.500 | 0.75 |
| | Total | 200.000 | 100.00 |

### Brief Manufacturing Process:

### 1.0 Sifting:

1.1 Sift mannitol and starch together through a suitable mesh.
1.2 Sift talc, colloidal silicon dioxide together through a suitable mesh.
1.3 Sift sodium stearyl fumarate through a suitable mesh.

### 2.0 Binder Solution:

2.1 Dissolve desmopressin acetate and copovidone in dehydrated alcohol with stirring.

### 3.0 Granulation:

3.1 Load the material of step no. 1.1 into the rapid mixer granulator and mix.
3.2 Granulate the dry mix of step no. 3.1 with binder solution of step no. 2.1.
3.3 Use additional dehydrated alcohol if required to get satisfactory wet granular mass.

### 4.0 Drying:

4.1 Dry the wet mass of step no. 3.3 in dryer.

### 5.0 Sifting and Milling:

5.1 Sift the dried granules of step no. 4.1 through a suitable mesh.
5.2 Mill the over size granules of step no. 5.1 and pass the milled granules through a suitable mesh. Repeat the process, till the entire material pass through a suitable mesh and mix all the sifted granules.

### 6.0 Blending:

6.1 Load the granules of step no 5.2 in blender.
6.2 Add sifted ingredient of step no. 1.2 to the blender and blend for suitable period of time.

### 7.0 Lubrication:

7.1 Add sifted ingredient of step no. 1.3 to the blender and lubricate for suitable period of time.

### 8.0 Compression:

8.1 Compress the blend of step no. 7.1 using rotary compression machine.

1. A process for the preparation of Desmopressin comprising the steps of:
a) swelling of a resin having a Fmoc group in a solvent,
b) deprotecting the Fmoc group,
c) anchoring a first protected terminal amino acid to the resin,
d) capping the resin obtained in step c),
e) selectively deprotecting the amino group of the anchored terminal amino acid,
f) coupling a carboxyl terminus of a next N-protected amino acid to the amine group,
g) repeating steps e) and f) to form a peptide sequence,
h) cleaving the peptide sequence from the resin and optionally deprotecting functional protecting groups using a cocktail mixture to obtain a dithiol derivative,
i) optionally purifying the dithiol derivative obtained in step h),
j) oxidizing the dithiol derivative to form a disulphide bridge to obtain crude Desmopressin,
k) passing the crude Desmopressin through an ion exchange resin,
l) purifying the crude Desmopressin to give pure Desmopressin acetate.

2. The process according to claim 1, wherein the resin selected is Rink amide (1.1 mmol/g).

3. The process according to claim 1, wherein the cocktail mixture is selected from a group comprising of TFA/Phenol/Thioanisole/DCM/water/TIPS in the preferred volumes of 80%/5%/3.33%/13.33%/3.33%/5% respectively or 80%/5%/3.5%/3.5%/8% respectively.

4. The process according to claim 1, wherein in step i) the purification is performed by at least one of precipitation and recrystallization, and the solvent or solvent mixture used for recrystallization is selected from ethyl acetate, methanol, ethanol, isopropanol and mixtures thereof.

5. The process according to claim 1, wherein the dithiol derivative is subjected to oxidation using a catalytic amount of iodine in methanol, air oxidation or K₃Fe(CN)₆ and the concentration of the dithiol derivative for oxidation is 1 g per 400 mL of methanol.

6. The process according to claim 1, wherein in step i) the crude Desmopressin is purified by passing through a weak anion exchange resin, evaporating methanol and precipitating with a solvent.

7. The process according to claim 6, wherein the crude Desmopressin is purified by a preparative HPLC method in the presence of a buffer and methanol as eluting agents and isolating pure Desmopressin acetate.

8. The process according to claim 6, wherein the purification of Desmopressin is carried out by preparative HPLC using a C-18 or C-8 column with 100 A° or 120 A° utilized on a reverse phase.

9. A pharmaceutical composition prepared by the process of claim 1 comprising pure Desmopressin compound and an excipients.

10. (canceled)

11. (canceled)

12. A process for the oxidation of a dithiol derivative for the preparation of Desmopressin comprising the steps of:
a) dissolving the thiol derivative to form a reaction mass,
b) adjusting the pH of the reaction mass to 8.0 with ammonia,
c) subjecting the reaction mass to oxidation, and
d) thus isolating the Desmopressin.

13. The process according to claim 12, wherein a reagent employed for oxidation is selected from iodine, K₃Fe(CN)₆ and exposure to air.

14. A process for the purification of Desmopressin by HPLC comprising the use as an eluent, of a mixture comprising one or more hydrocarbons, one or more alcohols and acetonitrile.

15. The process according to claim 14, wherein the purification of Desmopressin is carried out by preparative HPLC using a C-18 or C-8 column with 100 A° or 120 A° utilized on a reverse phase in the presence of a buffer and methanol as eluting agents to isolate pure Desmopressin acetate.
